(19) United States
(12) Patent Application Publication
(10) Pub. No.: US 2004/0029131 A1
(43) Pub. Date: Feb. 12, 2004

(54) Scanning kelvinmicroprobe system and process for biomolecule microassay

(76) Inventors: Michael Thompson, Toronto (CA); Larisa-Emilia Cheran, Toronto (CA); Mark McGovern, Mississauga (CA)

Correspondence Address: BORDEN LADNER GERVAIS LLP, WORLD EXCHANGE PLAZA, 100 QUEEN STREET SUITE 1100, OTTAWA, ON, K1P 1J9, CA

(21) Appl. No.: 10/296,508

(22) PCT Filed: May 18, 2001
(86) PCT No.: PCT/CA01/00716

(30) Foreign Application Priority Data
May 24, 2000 (CA) 2309412

(51) Int. Cl. C12Q 1/68; G01N 33/53; G21K 4/00; G03C 5/16; G01N 23/04

(52) U.S. Cl. 435/6; 435/7.1; 250/580

(57) ABSTRACT

There is provided a system and process for detecting biomolecular interaction on a substrate having a biomolecule immobilized on a surface of the substrate. The system and process incorporate a scanning Kelvin microprobe (SKM) capable of analyzing surface topography as well as a contact potential difference image signal. Also provided is the use of SKM in measuring and analyzing biochemical molecular interactions between a probe bound to the surface of the substrate, and a target suspected to be present in a liquid sample. One of the probe and target combination is a biomolecule such as a nucleic acid, a polypeptide, or a small molecule, and an antibody antigen combination may be used.

### FIELD OF THE INVENTION

This invention relates to a system and process for analysis of a substrates using a scanning Kelvin microprobe (SKM), and more specifically, to a system and a process incorporating SKM for analysis of biomolecule interactions on a substrate surface.

### BACKGROUND OF THE INVENTION

The Kelvin method for the measurement of work function can be employed for the analysis of a wider range of materials, at different temperatures and pressures, than any other surface analysis technique. Work function is a very sensitive parameter which can reflect imperceptible structural variations, surface modification, contamination or surface-related processes. The method is now regaining popularity¹⁻⁴ as a powerful technique because of its inherent high surface sensitivity, high lateral resolution due to the availability of nanometric precision-positioning systems, and improved signal detection devices. Unlike many other methods, the measurement of work function does not depend on an estimate of the electron reflection coefficient on the surface. Moreover, the technique does not use high temperature, high electric fields, or beams of electrons or photons. Being a non-contact and non-destructive method, it does not pose the risk of desorbing or removing even weakly-bound species from the surface. Furthermore, the Kelvin method is a direct measurement method requiring only a simple experimental set-up with no sample preparation.

When an electron is removed from a point within a material, the total change of thermodynamic free energy of the whole system is the difference between the change of the electrochemical potential of that material and the change of the electrostatic potential of the electron. If the electron is removed from a surface to a point in a vacuum, far from the outside surface so the surface forces have no more influence on the electron, this change of free energy is called the work function of that surface. The corresponding change when the electron is removed to another material that is in intimate electrical contact and thermal equilibrium with the first material, is called the contact potential difference (CPD). For example, when two different conductors are first brought into electrical contact, free electrons flow out of the one with the higher electrochemical potential (i.e., Fermi level) into the other conductor. This net flow of electrons continues until equilibrium is reached when their electrochemical potentials have become equal. The metal of higher work function (having originally a lower electrochemical potential) acquires a negative charge, the other conductor being left with a positive charge. When the whole system reaches thermodynamic equilibrium, the resulting potential difference is the CPD and is equal to the difference between their work functions.

In order to measure the CPD it is necessary to connect the conductors. A direct measurement with a voltmeter included in the circuit is not possible, since the algebraic sum of all the CPDs in the circuit is zero. Thus; CPD must be measured in an open circuit i.e., using a dielectric such as a vacuum or air between the conductors.

The Kelvin method is based on a parallel plate capacitor model: a vibrating electrode suspended above and “parallel” to a stationary electrode. The sinusoidal vibration changes the capacity between plates, which in turn, gives a variation of charge generating a displacement current, the Kelvin current, proportional to the existing CPD between the electrodes.

The last century witnessed a continuous process of improving and modification of the Kelvin probe in order to adapt it for particular applications⁵⁻¹⁰. The probe has been used in surface chemistry investigations, surface photo voltage studies, corrosion, stress, adsorption and contamination studies and was adapted for measurements in liquids, at high temperatures, in ion or electron emitting samples or in an ultra high vacuum environment¹¹⁻¹⁵. The problem of conducting measurements at the micrometer and sub-micrometer level has been overcome with the advent of SKM format which offers a new and unique tool to image the electrical potential on surfaces at the micrometer and sub-micrometer level. It has also been possible to develop an SKM instrument that is capable of generating both CPD and surface topographical images in tandem¹. Such equipment not only provides an electrical image of a surface, but also generates a truly tandem topographical image. Accordingly, electrical information can be integrated fully with chemical and morphological details, an extremely valuable feature for the users of the surface characterization technologies.

However, to measure the CPD on a small scale with high precision it is necessary to control closely the distance between the tip and the sample. This has been initially achieved¹ by processing the harmonics of the Kelvin current. However, this approach leads to instability and is unreliable.

### SUMMARY OF THE INVENTION

It is an object of the present invention to obviate or mitigate at least one disadvantage of previous processes and systems methods for applying SKM for biochemical analysis of microassays, for example analysis of nucleic acids, proteins, and small molecule interaction.

The invention provides a scanning Kelvin microprobe system for analyzing a biomolecular interaction on a surface of a substrate, said surface being capable of interacting with a biomolecule, the system comprises: a tip with a predetermined work function for exploring the surface, and for extracting Kelvin current from the local capacitor formed between the tip and the substrate; a scan table for placing the substrate thereon; a micropositioner for moving the scan table in x and y directions; a piezoelectric translation stage attached to the scan table for moving the substrate in the z direction for maintaining a constant substrate-tip distance; a charge amplifier for converting the Kelvin, current extracted by the tip into a voltage; a first lock-in amplifier tuned at a first frequency for measuring the voltage and generating a contact potential difference image signal; a second lock-in amplifier tuned at a second frequency for monitoring substrate-tip distance and for generating a topographic image signal, the second frequency being above the first frequency; and a controller for controlling the micropositioner.

Further, the invention provides a process for analyzing a biomolecular interaction on a surface of a substrate using a scanning Kelvin microprobe system, the surface being capable of interacting with a biomolecule, the process comprising the steps of: placing a substrate on a scan table; exploring a surface of the substrate with a tip having a predetermined work function; extracting Kelvin current from a local capacitor formed between the tip and the substrate; amplifying the Kelvin current extracted by the tip; measuring the Kelvin current and generating a contact potential difference signal using a first lock-in amplifier tuned at a first frequency; and monitoring distance between the substrate and the tip and generating a topographic image signal using a second lock-in amplifier tuned at a second frequency, the second frequency being above the first frequency.

The process for analyzing an interaction between a probe and a target using a scanning Kelvin microprobe system described herein may, more specifically, comprise the steps of: immobilizing a probe on the surface of a substrate; subjecting the probe to a first scanning Kelvin microprobe analysis; exposing the probe to a composition suspected of containing the target; subjecting the substrate to a second scanning Kelvin microprobe analysis; and comparing the results of the first and second scanning Kelvin microprobe analyses to determine interaction between the probe and the target.

From one aspect, the present invention provides applications of the scanning Kelvin microprobe (SKM) technology to the investigation of the immobilization of biochemical macromolecules such as proteins, DNA, RNA, DNA/DNA, DNA/RNA, oligonucleotides, or protein/nucleic acid and antigen/antibody pairings on various substrates. These biological moieties carry significant differences in charge. The latter, in turn, can be influenced by a number of important factors such as specific molecular reactions and tertiary structure. The present invention involves the study of the electrostatics of a biochemical moiety attached to a substrate, herein referred to interchangeably as a “probe”, by application of SKM technology to the multiplexed scanning of biochemical domains on substrates. Such analysis of biochemical microassays can be performed at a much higher spatial resolution than the existing fluorescence confocal microscopy technique.

One of the applications of the scanning Kelvin microprobe (SKM) technology is in investigating an immobilized biochemical macromolecule or probes such as proteins, DNA, RNA, DNA/DNA, DNA/RNA, oligonucleotides, or protein/nucleic acid, small molecules, and antibody/antigen interaction on various substrates. These biological moieties carry significant differences in charge which, in turn, can be influenced by a number of important factors such as specific molecular reactions and tertiary structure. There are few studies of the electrostatics of biochemical moieties attached to a substrate. However, it is apparent that the application of SKM technology lies in the multiplexed scanning of biochemical domains on substrates. Such analysis of biochemical microarrays can be performed at a much higher spatial resolution than the existing fluorescence confocal microscopy technique. Coupled with advances on the direct attachment of oligonucleotides and high resolution robotic printing, SKM allows the analysis of nucleic acid duplex formation at extremely high array density.

A preferred embodiment of the invention couples SKM application with advances on the direct attachment of oligonucleotides and high resolution robotic printing. In this way the SKM utilization according to the invention leads to, for example, the analysis of nucleic acid duplex formation at extremely high array density, as demonstrated below in experiments on surface-bound macromolecules.

The SKM system employed according to the invention uses a higher frequency (sample-tip capacitance detection) to control the sample-tip distance, thus, making the process stable and reliable. The automated monitoring of the contact potential and topography was achieved using 2 lock-in amplifiers tuned respectively on the vibrational frequency and on the capacitance-detection frequency. This means that the monitoring of the sample-tip distance is no longer achieved by processing the harmonics of the CPD signal as taught by the prior art, but by measuring the sample-tip capacitance at a frequency above the vibrational frequency. This approach solves the instability and unreliability problems that affect the prior art. The current prototype has a superior lateral resolution achieved by employing amplifiers capable of detecting low-level currents extracted by extremely fine tip probes having an apex radius of curvature below 100 nm. The invention advantageously comprises a data acquisition and imagining system. Further, the null-condition measurement according to the invention avoids the strong electric fields that affect the surface of the specimens in prior art apparatuses. This is also an advantage over the force microscopes operating in Kelvin mode that develop extremely high local electrical fields (10⁹ V/m range), thus affecting both the local distribution of charges and the spatial conformation of the investigated molecules.

The scanning instrument employed in the invention is capable of CPD measurement to a lateral resolution of 1 micron and can display a resolution of 1 mV. The invention fullfils a long-standing need for high resolution measurements for biochemical microassays. With this instrument, it is now possible to generate new knowledge and applications in surface physical chemistry and material characterization, as particularly required in biochemical microassays. Advantageously, the inventive technique is non-destructive. The technology has applications in biochemical microassays relating to chemical analysis, photochemical studies and biosensor technology.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures.

FIG. 1 is a diagrammatic illustration of the measurement of contact potential difference CPD according to the invention.

FIG. 2 is a schematic drawing of the instrument used in exemplary embodiments of the invention described herein.

FIGS. 3A and 3B are tandem topographical and CPD images of a bare silicon wafer used as an oligonucleotide substrate in Example 1.

FIG. 4 is a CPD image of an oligonucleotide (Fi) attached to an Si surface according to Example 2.

FIG. 5 is a CPD image of an F₁:F₂ duplex attached to an Si surface according to Example 3.

FIG. 6 is a confocal fluorescence microscope image of a DNA microarray according to Example 4.

FIG. 7 is and a SKM image of a selected area shown on the DNA microarray image of FIG. 6.

### DETAILED DESCRIPTION

The instrument according to the invention can be used for characterization and analysis of surfaces of materials, based on the variation of work function values associated with interfacial properties. This variation of work function is determined by the measurement of contact potential using the Kelvin probe method. This technique is founded on a parallel plate capacitor model, where one plate possesses a known work function and is used as a reference. The material with unknown work function represents the other plate. An embodiment of the present invention is an SKM instrument that is capable of CPD measurement to a lateral resolution of 1 micron and displays a resolution of 1 mV. A unique feature of the instrument is its capability to generate both CPD and surface topographical maps in a tandem fashion reliably. Further, the method is non-destructive.

According to the invention, the scanning Kelvin microprobe can be used as a unique tool for investigating physics and chemistry of surfaces. One application is in the investigation of interfacial phenomena in biosensor technology, especially the electrostatics of DNA on surfaces. The SKM can scan surfaces of biomaterials, including biosensors, for the spatial location of moieties such as proteins and oligonucleotides. These biological species carry significant charge which can lead to highly significant differences in surface potential related to specific molecular reactions. This, in turn, leads to the possibility of the multiplexed scanning of biomolecular interactions such as attachment of single and double DNA strands to different substrates such as glass, mica, silicon, chromium, and complementary duplex strand formation.

The substrate to be analyzed according to the invention can be any such substrate capable of interaction with a biomolecule. Such substrates may have a probe molecule immobilized or in any other way bound thereto. The substrate may be a biosensor, a biochemical microarray, such as a biochip, a thin film or monolayer including material capable of interacting with a biomolecule.

The scan table is movable by any requisite amount so as to allow exploration of a sample surface, for example by about 200 nm in either the x or y direction. The scan table may optionally have course and fine adjustment, for example, coarse adjustment of about 100 nm and fine adjustment of about 4 mm.

Kelvin current is generated when two electrodes or plates are brought in electrical contact with a measuring device and the Fermi levels of two electrodes equalize. The Kelvin current is a measure of contact potential difference (CPD) of the two electrodes.

Contact potential difference is the difference between the work functions of two materials in contact. Measurement of the CPD thus affords a method of measuring work function differences between materials. In order to measure the CPD it is necessary to connect the materials. A direct measurement, for instance with a voltmeter, requires a circuit shortened by a measurement device. However, in a closed circuit CPD cannot be measured directly, as the sum of the three interfacial differences would be zero, except for the case where the interfaces have different temperatures. Thus, CPD is measured in an open circuit, for example using a dielectric medium such as a vacuum or air.

Work function is the work required to extract an electron from the Fermi level to infinity.

A local capacitor is formed between the tip and the substrate. The tip extracts Kelvin current from this local capacitor. A capacitor is capable of storing charges, formed by arrangement of two conductors or semiconductors (electrodes or plates) separated by a dielectric medium, such as air or a vacuum.

Capacitance is the property of a material whereby it stores electric charge. If an isolated conductor is placed near a second conductor or a semiconductor but is separated from it by air or some other insulator, the system forms a capacitor. An electric field is produced across the system and this field determines the potential difference between the two plates of the capacitor. The value of the capacitance of a given device is directly proportional to the size and shape (area) of the electrodes and the relative permittivity of the dielectric medium, and inversely proportional to the distance between the two plates. According to the invention, the tip and the substrate surface act as the two plates of the capacitor, and air or another gaseous medium, such as nitrogen or argon, is used as the dielectric medium.

The tip of the scanning Kelvin microprobe system according to the invention is used to scan a substrate surface and to extract Kelvin current from the capacitor formed between the tip and the substrate surface. The tip can be made of any suitable material with a known work function, for example, tungsten. In one embodiment of the present invention, the tip is a guarded microelectrode having the apex radius of curvature less than about 100 nm, and optionally in the range of about 50 nm.

The substrate is placed on a scan, table, which is capable of moving in the x, y, and z directions. The micropositioner provides a means for moving the scan table in x and y directions, and expediently comprises a computer-related device. A translation stage is used to move the scan table in z direction, that is upwardly (closer to) or downwardly (further from) the tip. By the terms upwardly and downwardly, vertical direction is not implied, although the z direction may optionally be in the vertical direction. In one embodiment of the invention, the translation stage is a piezoelectric translation stage. Particularly, the translation stage can be controlled by piezoelectricity.

The charge amplifier, which may be a series of amplifiers, such as a pre-amplifier plus a charge amplifier, allows magnification of an input electrical signal for output. In one embodiment of the present invention, the charge amplifier is an ultra low noise charge amplifier.

The lock-in amplifiers are detectors that respond only to an input signal having a frequency synchronous with the frequency of a control signal. A lock-in amplifier can be used to detect a null point in a circuit. According to the present invention, a first lock-in amplifier and a second lock-in amplifier are used. Each is tuned to a separate frequency, and the frequencies are non-interfering. The first frequency can be any from about 1 to about 20 kHz, while the second frequency can be any from about 100 to about 500 kHz. The second frequency is above the first frequency, so that the two frequencies are non-interfering.

A data acquisition system may be incorporated into the scanning Kelvin microprobe system, for acquiring the contact potential difference image signal and said topographical image signal. A controller may be used for the system, comprising software capable of opening a file, initializing a card and a motor, starting the first and the second lock-in amplifiers, bringing the tip down, scanning the substrate surface, bringing the tip up, writing data in a file, and closing the file. The controller may also be incorporated within a hardware component.

The system according to the invention can be used for characterization and analysis of surfaces of biomaterials, based on the variation of work function values associated with interfacial properties. This variation of work function is determined by the measurement of contact potential using the Kelvin probe method. This technique is founded on a parallel plate capacitor model, where one plate possesses a known work function and is used as a reference, while the material with unknown work function represents the other plate. An embodiment of the present invention is an SKM instrument that is capable of CPD measurement to a lateral resolution of 1 micron and displays a resolution of 1 mV. A unique feature of the instrument is its capability to generate both CPD and surface topographical maps in a tandem fashion reliably. Further, the method is nondestructive.

The scanning Kelvin microprobe (SKM) according to the invention can be used as a unique tool for investigating the physics and chemistry of surfaces. The instrument has application in a number of fields, including but not limited to, chemical sensors and biosensors, biocompatibility, coatings, adsorption, contamination, microarrays, and biochips.

One application of the SKM is in the investigation of interfacial phenomena in biosensor technology, especially the electrostatics of DNA on surfaces. The SKM can scan surfaces of biomaterials, including a biosensor, for the spatial location of moieties such as proteins and oligonucleotides. These biomaterials carry a significant charge which can lead to highly significant differences in surface potential related to specific molecular reactions.

The SKM technology according to the invention is also a powerful tool for the study of surface morphology, structural variations, surface modification, electrochemical surface reactions and the local determination of various surface parameters.

The inventive system and process can be used for analyzing an interaction between a probe and a target. A probe molecule is immobilized or otherwise bound to a substrate surface using technology known in the art. The target is a biomolecule suspected to be present in a liquid medium, which can be exposed to the substrate-bound probe, and have a physical interaction therewith. Should the target biomolecule bind to the probe, this effects the subsequent SKM reading, and thus binding can be detected. Initially, a probe is immobilized on the surface of a substrate after which the probe/substrate combination is subjected to a first scanning Kelvin microprobe analysis. Alternatively, this scanning may be done with a standard, or using a stored reference within a memory unit. The probe/substrate is then exposed to a composition suspected of containing the target, for example, and aqueous biological solution such as serum, plasma, a tissue sample, suspended cells, etc., after which the exposed substrate undergoes a second scanning Kelvin microprobe analysis. Of course, if the first analysis was conducted by way of referring to a reference file or standard, this post-exposure scanning would be the first actual analysis of the particular substrate in use. The results of the first and second scanning Kelvin microprobe analyses are then compared to determine interaction between the probe and the target, for example, if there is binding of the target to the probe. This allows quantitative and qualitative analysis of a biological solution.

In such a process, either one of the probe and the target is a nucleic acid, a polypeptide, or a small molecule. In the case where a small molecule is used, the other of the probe or the target may be any molecule capable of interaction with such a molecule. For example, an enzyme may be a target, and the probe may be a substrate effective with that enzyme. As a further example, the probe and the target combination can be an antigen/antibody combination, with either molecule being the target.

Development of an Improved Scanning Kelvin Microprobe System

The Kelvin method is based on the measurement of work function by a configuration consisting of a vibrating electrode suspended above and parallel to a stationary electrode. The sinusoidal vibration of one plate alters the capacity between the plates resulting in a Kelvin current, which is proportional to the existing CPD between the plates.

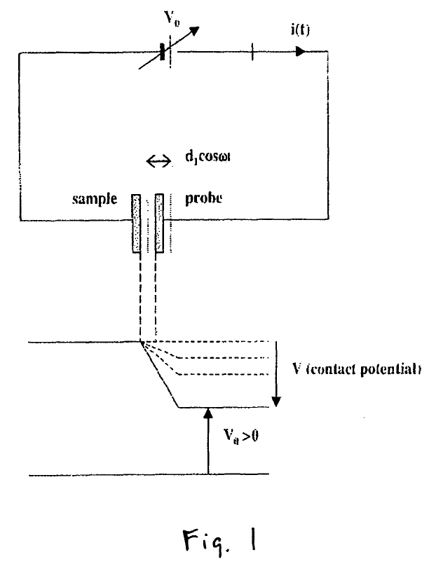

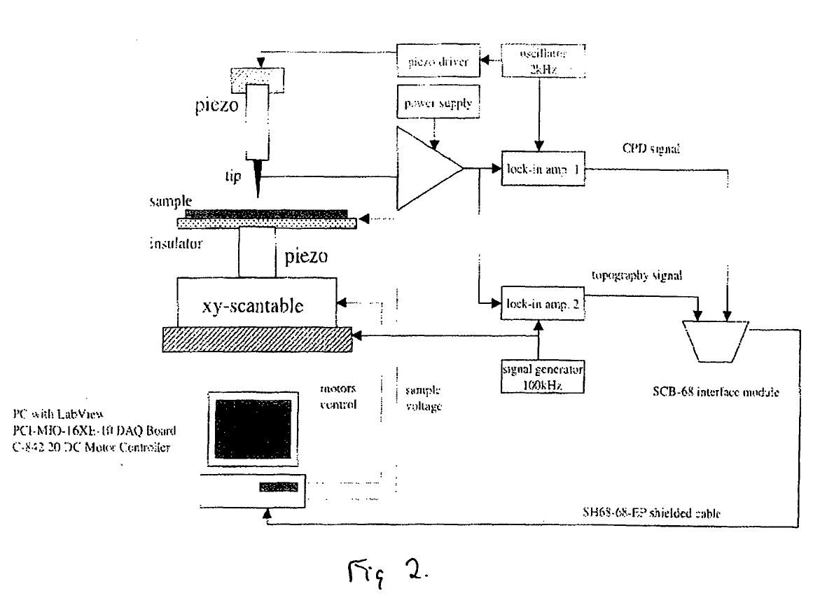

FIG. 1 shows the principle of the Kelvin method used in the present invention The instrument shown has a vibrating tip (50) made of material with a known work function such as tungsten, which explores, point by point, the surface of the sample (52), extracting the Kelvin current from the local capacitor formed under the tip. When a thermodynamic equilibrium is established, a CPD appears between the two “plates” as a voltage V, or contact potential and the capacitor is charged. Since V remains constant, but the distance between the tip and the sample changes, the charge on the plates changes too. The tip (50) has a sinusoidal vibration, so the separation distance between the plates is:

d(t)=d₀+d₁ cos ωt (1)

where d₀ is the rest position and d₁ is the amplitude of the vibration. The frequency of the vibration is set at f₁=2 kHz. The capacity is then:

$$C ( t ) = \frac{ϵ A}{d ( t )} = \frac{ϵ A}{{d}_{0} + {d}_{1} cos ω t} \qquad ( 2 )$$

wherein A is area of a plate, ε is the dielectric constant, and t is the time. An adjustable DC voltage source, V₀ (54) is inserted in the circuit (56). The capacitor charging process causes a current in the measurement device, the Kelvin current:

$$i ( t ) = \frac{ⅆ Q ( t )}{ⅆ t} = ( V + {V}_{0} ) \frac{ϵ ω {d}_{1} A sin ω t}{{( {d}_{0} + {d}_{1} cos ω t )}^{2}} \qquad ( 3 )$$

If the contact potential is compensated by the variable voltage source (54), there will be no current flowing in the circuit (56). This compensation is detected as a null-condition by a sensitive lock-in amplifier.

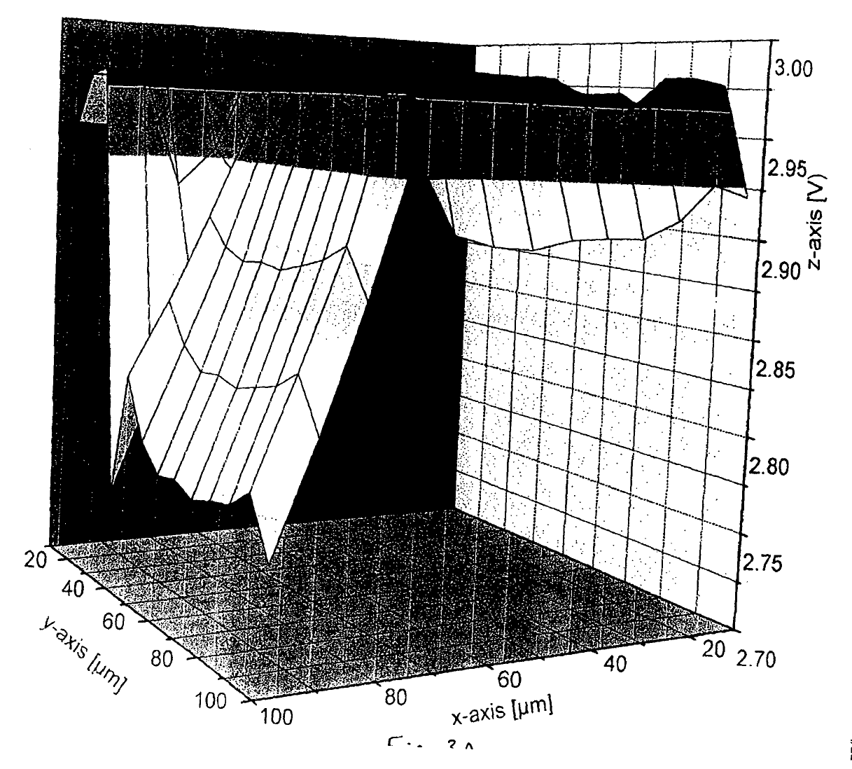

FIG. 2 presents a schematic diagram of the instrument of an embodiment of the present invention. The system comprises of the following components: a scanning system having a tip (60), tip holder (62), piezoelectric element (64), piezoelectric element driver (66); vibrational frequency generator or oscillator (68), insulator (70), and a scan table (72) controlled by a micropositioner; a sample-tip distance control unit having a piezoelectric translation stage (74) and a capacitance-detection frequency generator; a measurement system having an ultra low-noise charge amplifier (76), a first lock-in amplifier (78) for measuring the voltage and generating a contact potential difference image signal, a second lock-in amplifier (80) for monitoring sample-tip distance and for generating a topographic image signal, vibrational frequency generator, and capacitance-detection frequency generator; a signal collection unit (82) having an interface module for interfacing the measuring system with a data acquisition (DAQ) board installed inside the computer; and a computing device for controlling the system.

A sample is placed on the scan table. The scan table is movable in the directions of the x-axis and the y-axis in order to have the sample scanned. The position of the scan table is adjusted by a micropositioning system (Nanonics, Israel)) which moves the scan table in x and y directions with a coarse resolution of 100 nm (closed loop DC motor) and a fine resolution of 4 nm (closed loop PZT drive), respectively. The control of the micropositioning system is achieved by a motor controller board installed in the computer. A piezoelectrically driven translation stage is mounted on the top of the scan table. The stage moves along the z-axis in order to maintain a constant distance between the tip and the sample.

The tip is attached to the piezoelectric element via the tip holder. The frequency of the vibration, f₁, to vibrate the tip, is generated by a frequency generator (oscillator) and is then fed into the vibrating piezoelectric element (Topometrix, Calif., U-SA) through the piezoelectric driving amplifier (I.P. Piezomechanik, Germany).

The Kelvin current extracted by the tip is converted to a voltage and amplified by means of an ultra low-noise preamplifier and a charge amplifier (A250+A275, Amptek Inc. USA). This voltage is fed at the entrance of the two lock-in amplifiers.

The first lock-in amplifier (SR530, Stanford Research Systems, USA) is tuned at f₁ and used to obtain the CPD signal. The f₁ may range from 1-20 kHz. The output voltage of the CPD lock-in amplifier is returned to the probe in a feedback loop (not shown). For large enough values of the open loop gain, the contact potential value is given directly by the output voltage of the lock-in amplifier.

The distance between the sample and the tip is monitored via capacitative control at a frequency above the vibration frequency f₁. The f₂ may range from 100-500 kHz. A small AC voltage (100 mV at frequency, f₂=100 kHz) is added in the circuit and the resulting Kelvin current between the tip and the sample is detected by a second lock-in-amplifier (SR530, Stanzford Research Systems, USA) tuned to f2. The tip-sample capacitance is kept constant by returning the output signal of the second lock-in amplifier to the piezoelectric translation stage. This signal is also used to obtain the topographical image of the sample.

The data acquisition and signal processing is done with the data acquisition board (PCI-6110) installed in the computer. All electric cables are carefully shielded and a BNC 2120 interface module is used for connections. The BNC 2120 interface module is a connector module interfacing the measuring system with the DAQ (data acquisition) board installed inside the computer. It contains a function generator, BNC connectors for analog input channels, analog output, digital input/output

The system is controlled by a computing device having a PCI-6110 DAQ board (National Instruments), the motor controller C-842.20DC and the LabView programs (version 6I).

### EXAMPLES

The invention is further described, for illustrative purposes, in the following specific Examples. General methodology having application to all examples is described herein below.

Reagents. The following chemicals were obtained from Aldrich and used as received: ω-Undecanoyl alcohol 98%, 6,6′-dithiodinicotinic acid, trifluoroacetic anhydride 99%, hydrogen hexachloroplatinate (IV) 99.99%, octadecyltrichlorosilane (OTS), trichlorosilane 99%, 3-mercaptopropyltrimethoxysilane (MPS), N-bromosuccinimide (NBS),1,1′-azobis-(cyclohexanecarbonitrile)(ACN), and dimethylformamide-sulfurtioxide complex. Various common solvents and chemicals were obtained from BDH and used without further treatment unless otherwise indicated as follows. Dichloromethane and acetonitrile, toluene and pyridine were distilled over P₂O₅, Na and KOH, respectively, and benzene and DMF were dried over molecular sieves before use.

Silicon wafers obtained from International Wafer Service were supplied approximately 0.4 thick and were polished on one side to a mirror finish. They were cut to a size of about 1×1 cm using a diamond-tipped pencil.

Syntheses. 1-(thiotrifluoroacetato)-11-(trichlorosilyl)-undecane (TTU) was synthesized and characterized as described previously¹⁶⁻¹⁹. The sodium salt of 2.5-bis (bromomethyl) benzensulfonate (BMBS) was produced by bromomethylation of p-xylene followed by conversion to the sulfonate (sodium salt) with DMF-sulfurtrioxide reagent and NaOH.

Oligonucleotide syntheses of the following thiolated sequences 5′-HS-C6-TATAAAAAGAGAGAGATCGAGTC-3′ (F₁) and its single strand, un-thiolated complement (F₂), were performed using standard CE phosphoroamidite chemistry with conventional Applied Biosystems Inc. reagents. In order to produce the thiol-group containing oligonucleotide, an iodine solution was employed in conjunction with 3′-thiol modification cartridges (Glen Research). The oligonucleotides were purified using standard procedures with Poly-Pak cartridges purchased from Glen Research. The final products were checked for purity by HPLC and stored in 20% acetonitrile, in polypropylene vials. Solutions of F₁ were treated with a ten-fold excess of BMBS at neutral pH in order to produce an oligonucleotide-linker complex.

Procedures. Silicon surfaces were silanized in a dry box for 2 hours with 2 ml of a 10⁻³ M solution in dry toluene of a mixture of 30% TTU/70% OTS. The TTU coated wafers were treated with hydroxylamine in water (pH 8.5) for 2 hours to effect deprotection of the thiol group. The F₁ oligonucleotide was attached to the surface via the linker BMBS as described elsewhere¹⁹. Hybridization of the surface-bound oligonucleotide with its complementary strand was effected in pH 7.5 buffer at room temperature.

DNA microarray. A glass substrate containing partially-hybridized DNA associated with examination of the yeast genome through variable size DNA probes was obtained by donation. This microarray, produced by robotic printing, consisted of 6400 probe domains of 150×150 μm dimension spaced by 200 μm gaps.

Surface immobilization of 25-mer oligonucleotides. The design and fabrication of biosensors capable of the detection of interfacial nucleic acid hybridization and interaction with small molecules such as drugs, regulatory peptides is an important area of study. This research activity requires the attachment of single strands of oligonucleotides to the device surface. A protocol extensively for achieving this involves nucleic acid-surface binding though interaction of chemisorbed neutravidin with biotinylated oligonucleotide. This method produces a surface nucleic acid density of only, at best, 1 pmol cm⁻² (compared to the maximum-possible, for single strands, of about 100 pmol cm⁻²)¹⁸. However, the sensitivity of device response can be enhanced by increasing nucleic acid surface density through silanization technology (on sensor chromium electrodes). The silane employed in the present experiments, to increase nucleic acid surface density, TTU, attaches to hydroxylated substrates by a self-assembly process to produce a near monolayer-like array of thiol functionalities (following de-protection of the sulfur-containing moieties). Dilution with OTS serves to minimize thiol-group cross linking interactions, and the use of a linking agent that forms disulfide bonds such as BMBS was found to optimize surface density of 11-mer oligonucleotides at about 50 pmol cm⁻² on silicon wafers¹⁹.

### Example 1

Surface Measurement and Analysis of Silicon Wafer by SKM

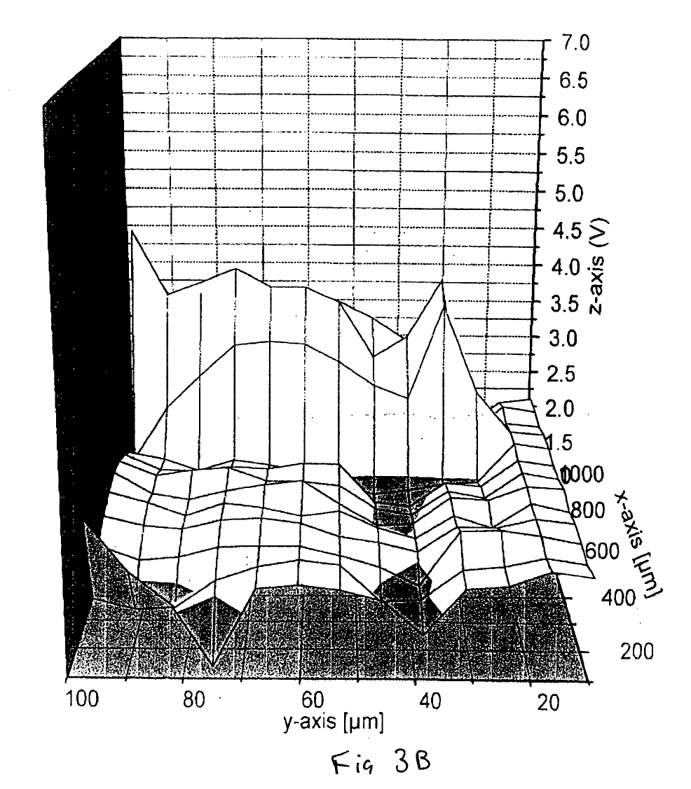

This experiment was conducted to obtain images that can serve as a control for any changes produced by subsequent surface chemical treatments. FIGS. 3A and 3B show the tandem topographical and CPD images obtained at 20 μm spatial resolution for the bare silicon wafer, respectively. The wafer was used for the immobilizing nucleic acids. With respect to the topographical image, the picture was recorded viewing from the y-axis in order to isolate an obvious fissure of depth about 800 nm (width at half-depth is 100 μm). Aside from this structure, which is likely related to scratching connected to a polishing protocol, the surface height variability is of the order of 300 nm (0.15 V). The image also exhibits fairly uniform “peaks” with a half height dimension of about 100 nm. These characteristics are expected from a substrate surface that is considered to be optically flat. The CPD image shows a quite narrow range of surface variability of approximately of 75 mV, which is likely connected to differences in the level of oxidation and/or contamination from adventitious carbon. Note that the features on terms of spatial characteristics reflect the same overall picture as shown for the topographical image.

### Example 2

Surface Measurement and Analysis of Oligonucleotides Attached to a Silicon Substrate

The immobilization of nucleic acids on biosensors and gene chips using TTU represents a new research area. The attachment of oligonucleotides to a silicon substrate was tested by employing the capabilities of the new SKM instrument.

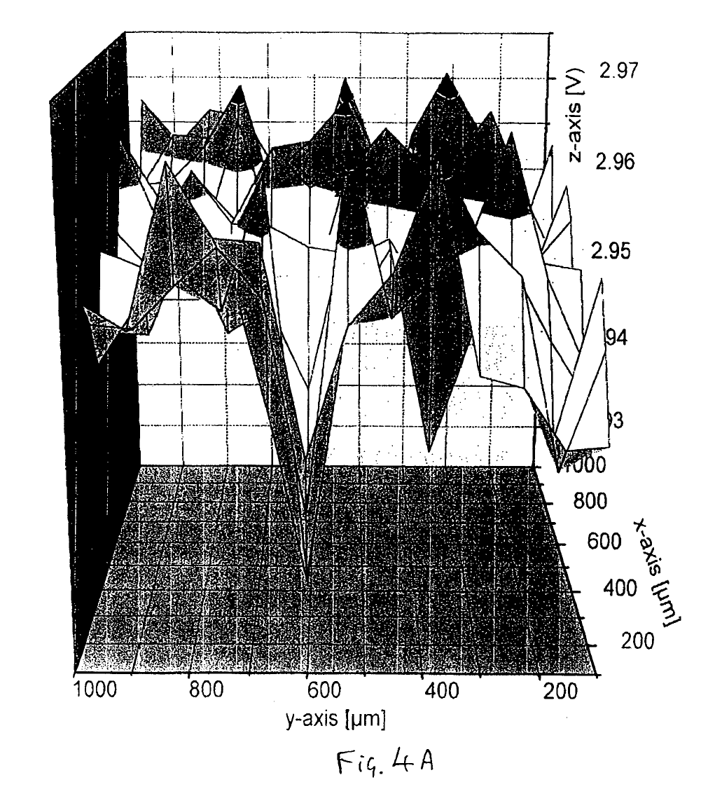

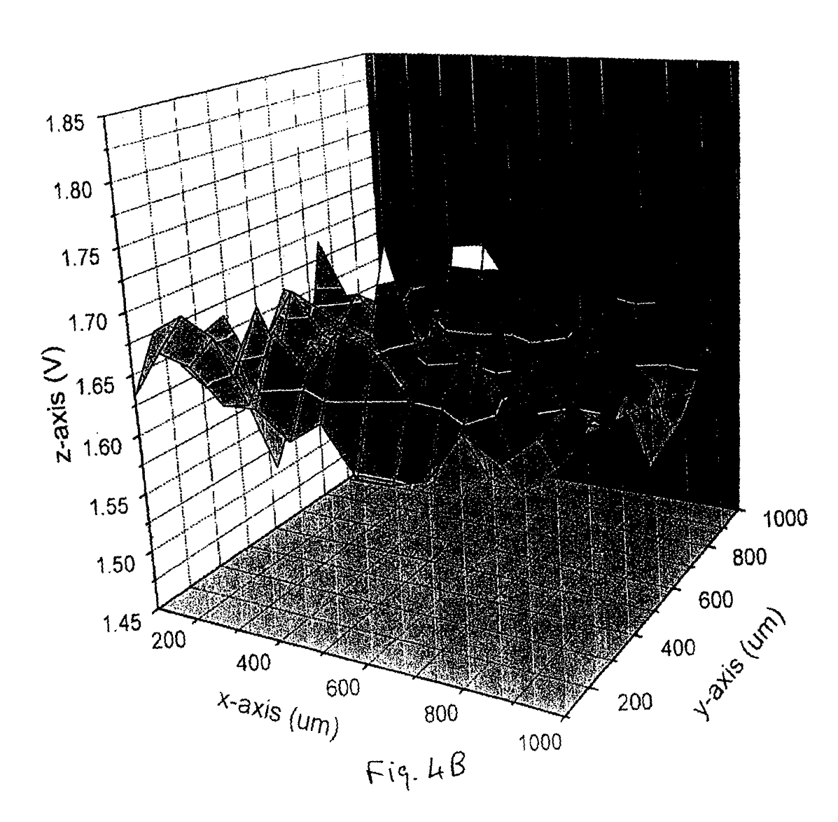

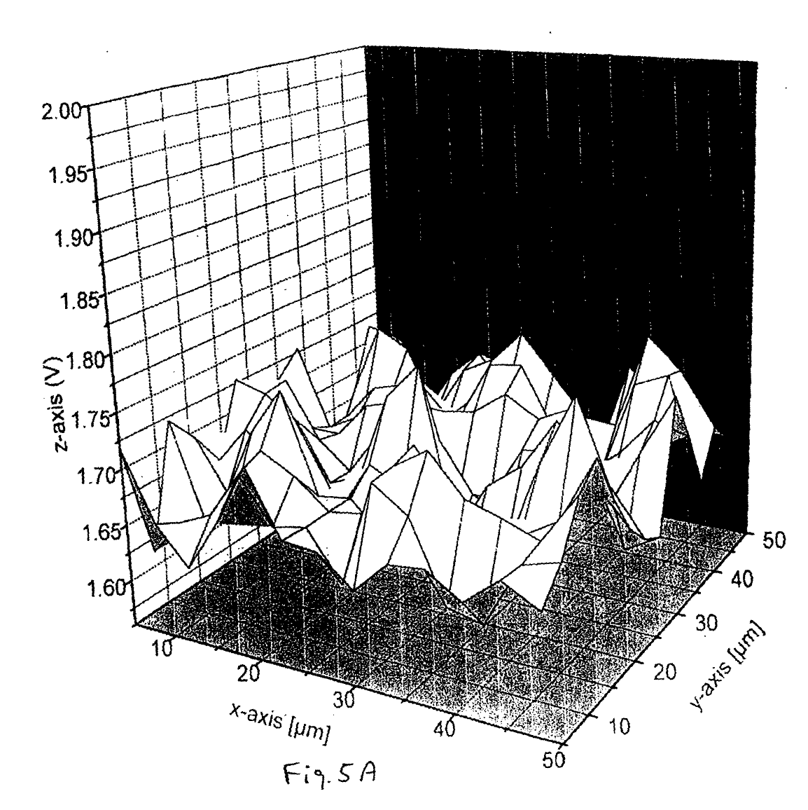

With respect to the use of Kelvin probe measurements to distinguish oligonucleotide and DNA duplex formation, the 25-mer probe, F₁, with BMBS linker in place, attaches to the de-protected TTU monolayer on the Si wafer through formation of a disulfide bond. Using this approach, the probe is disposed closer to the substrate surface at the 5′-end, whereas the 3′-terminus faces away from the interface. Experience has shown that the surface packing density attainable by this attachment protocol is of the order of 20 pmol cm^{-2.} This value implies that the surface density of attached nucleic acid is in the region of 1 molecule per 10 square nanometers. The precise orientation of the probe is unknown in terms of the air-to-solid interface. FIG. 4 shows the CPD image of Si surface-attached F₁ (1 μm resolution). The surface variability is in the range of about 100 mV with the mean value being 1.70 V. This represents a shift of approx. 80 mV per the average CPD value for the bare Si surface. There are “peaks” depicted in the image with widths at half-height of about 7 μm (spaced by 10 μm).

### Example 3

Surface Measurement and Analysis of Duplex Formation Between Oligonucleotides

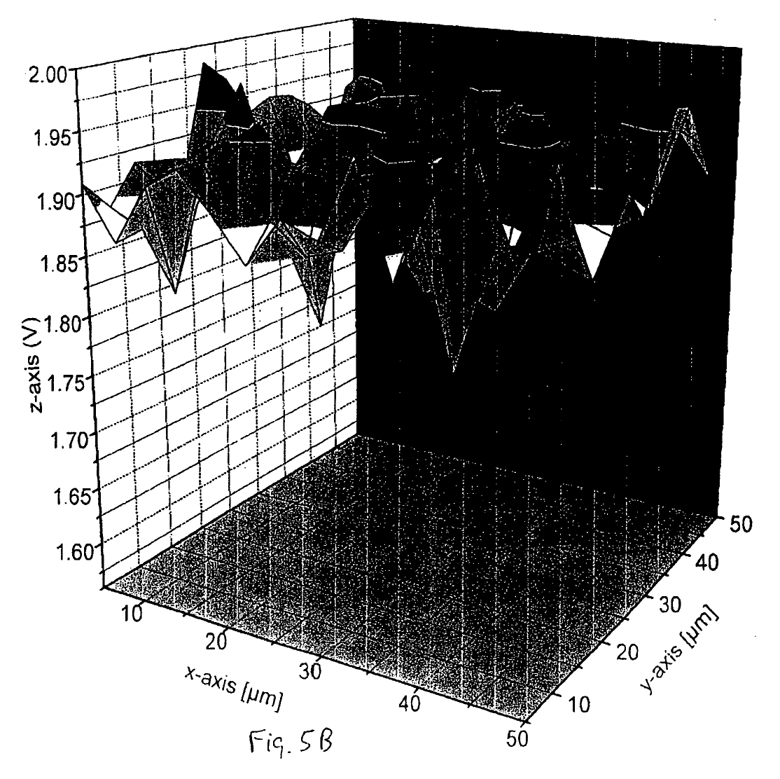

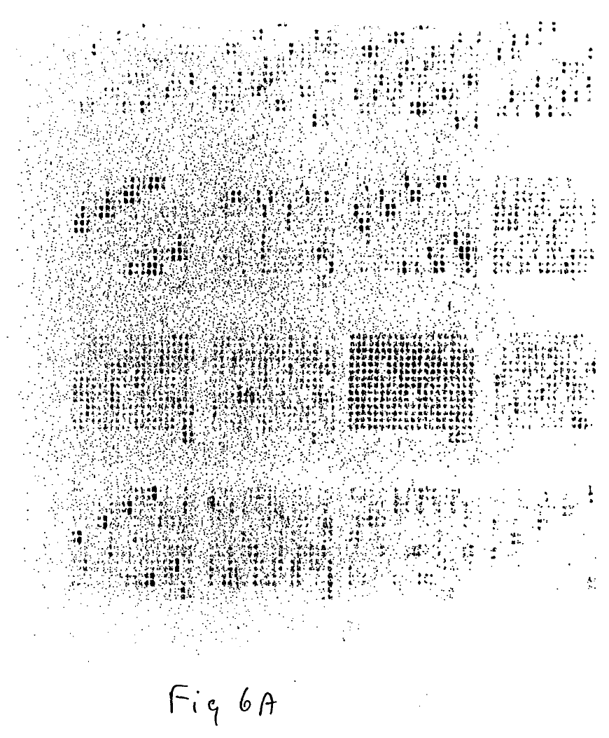

FIG. 5 shows the CPD image of the same surface for F₂ hybridized to F₁. The overall surface variability and features are much the same as for the single strand 25-mer attached to the substrate, but the CPD value has shifted upward by over 200 mV. This result clearly indicates that detection of duplex formation by the SKM is feasible. Since the attainable resolution in relative CPD value is 1 mV, this result implies that high discrimination of the level of duplex formation connected to mismatches is feasible.

### Example 4

Comparison of Measurement of DNA Microarrays by Fluorescence Microscopy and SKM

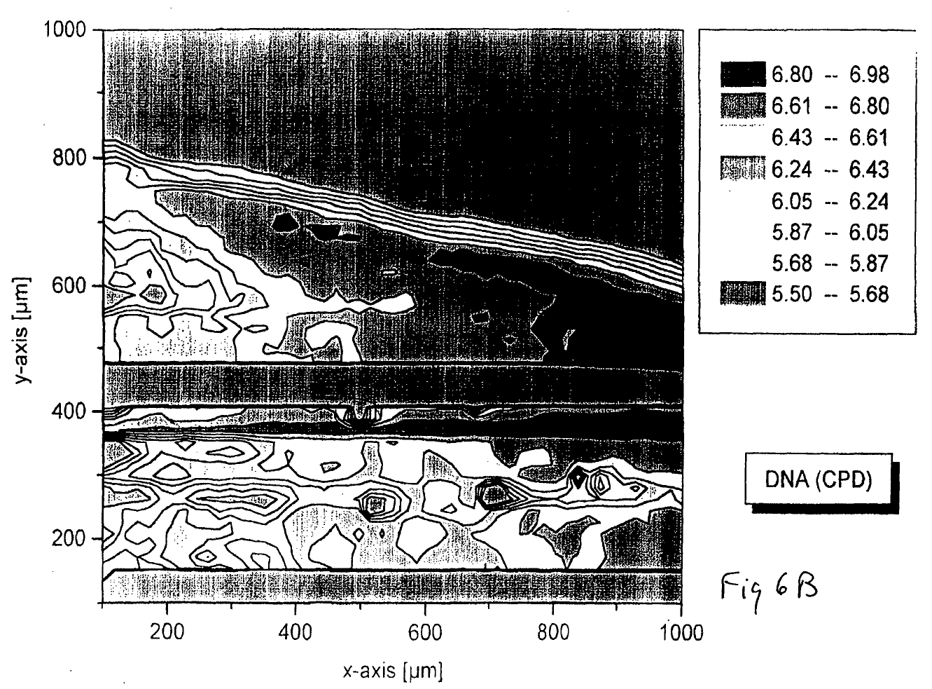

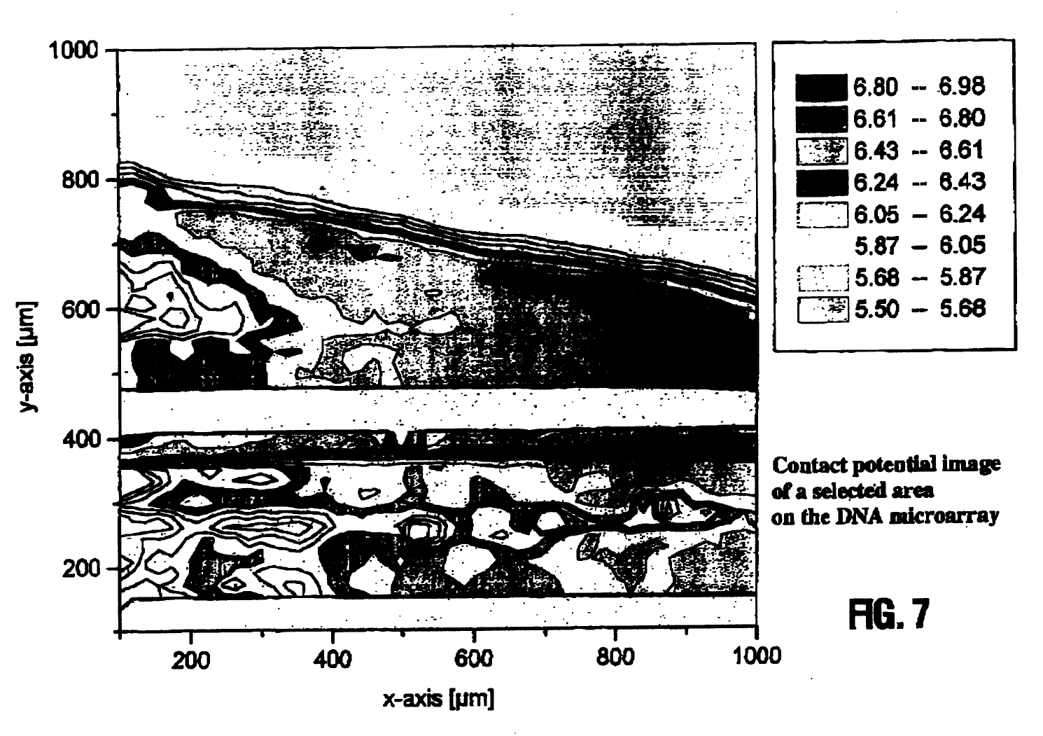

DNA microarrays were used to compare fluorescence microscopy and the SKM as detecting methods for DNA hybridization on gene chips. FIG. 6 shows a fluorescence image of typically hybridized probe domains and indicates the area of 5×5 points subsequently investigated by the SKM. A 20 μm lateral resolution was chosen this because a 1 μm or 100 nm resolution would be useless on the 150 μm DNA spots. A better resolution is, however, extremely appealing if a much higher deposition density of DNA strands is envisaged. FIG. 7 shows one of the lines with its 5 DNA islands spaced at 200 μm and some of the points above. The first 4 islands have the same CPD value situated in the range 5.5-5.68 V; the 5th island clearly presenting a higher CPD level around 6.2 V. Matrix transposition causes a reversion of the actual image. Taking this into account, the second line of the quadrant indicated in FIG. 6 matches the SKM line shown in FIG. 7. However, using an extremely accurate micropositioning device that will follow the exact pattern of DNA deposition (or alternatively replacement of the microfluidic deposition head by an SKM microprobe) one can assess directly DNA hybridization on microarrays, without using the time-consuming intermediate steps. This provides higher accuracy than is possible with present-day conventional fluorescence microscopy.

There exists the possibility of sample alteration due to the application of an electric field on the surface. Force microscopes operating in the Kelvin mode²⁰⁻²² require a large ac voltage modulation between tip and sample in order to obtain the desired sensitivity to variation in the contact potential signal: typically several volts modulation for a 1 mV sensitivity. For a sample-tip distance of 10 nm, the electric field generated can reach 109 V/m (for a 10 V modulation). Such strong fields affect the electrostatic conditions at the surface of the sample, as clearly observed by consecutive measurement made with the instrument described herein, first with force microscopy simulated conditions (with 10 V applied on the probe), second in normal operation (not exceeding 100 mV which are needed for obtaining the voltage modulation). The comparative experiment clearly shows an altered surface potential image due to the application of the strong electric field, both on CPD image and on topography. This means that aside from electrostatic alteration, some local alteration of spatial configuration of biomolecules deposited on surfaces also occurs. From this specific point of view, therefore, the SKM represents a serious alternative for conducting surface analysis.

The results reported herein indicate the advantages that SKM technology presents over conventional fluorescence microscopy for the detection of microarray duplex formation. The technique provides direct information, thus avoiding the necessity to employ tagging agents. Furthermore, much higher lateral resolution can be achieved compared to the spatial limits imposed by the use of light-based technology. This, in turn, leads to the possibilities of the analysis of microarrays at a much higher domain density, and for the characterization of the true homogeneity of layer structures with dimension on the order of 50-100 μm dimensions. At the present time, however, it is not possible to generate domain sizes down to the 1 μm level or lower because of the inherent limitations associated with spreading phenomena in robotic printing. There is no doubt that the photolithography-combinatorial synthesis of oligonucleotide arrays renders mm-sized structures as feasible, but this configuration, by definition, is restricted to the use of relatively short oligonucleotides (e.g. approx. 20 mers).

While specific methods of attachment of oligonucleotides to substrate have been described herein and used in the experimental examples, it is to be understood that this is by way of illustration, and the invention is not limited thereto. It is of general application to the detection of surface-bound DNA interaction with probe DNA, using SKM principles. For example it can be used to analyze nucleic acid—surface binding through interaction of chemisorbed neutravidin with biotinylated oligonucleotide¹⁸, and other similar binding systems. It can also be used generally with biochemical molecule-biochemical molecule interactions, not restricted to DNA hybridization, e.g. in determining potential drug receptor interactions and bindings.

References

1. L -E. Cheran, H -D. Liess, M. Thompson, The Analyst, 1999, 124, 961.

2. W. Nabhan, B. Eqer, A. Broniatowski and G. De Rosny, Rev. Sci. Instrum., 1997, 68 (8), 3108.

3. M. Schmidt, M. Nohlen, G.,Bermes, M. Bomer and K. Wandelt, Rev. Sci. Instrum., 1997, 68, 10, 3866.

4. M. Bomisch, F. Burmeister, A. Rettenberg, J. Zimmermann, J. Boneberg and P. Leiderer, J. Phys. Chem. B, 1997, 101, 10162.

5. W. A. Zissman, Rev. Sci. Instrum., 1932, 3, 367.

6. P. Craig and V.Radeka, Rev. Sci. Instrum., 1970, 41, 2, 258.

7. N. A. Surplice and R. J. D'Arcy, J.Phys.E: Sci. Instrum., 3, (1970), 477.

8. B. Ritty, F. Watchel, R. Manquenouille, F. Ott, J. Bonnet, J. Phys. E: Sci.Instrum., 15, 1982, 310.

9. I. D. Baikie, E. Venderbosch, Rev.Sci.Instrum. 62 (3), 1991, 725.

10. O. A. Semenikhin, L.Jiang, T. Iyoda, K. Hashimoto, A. Fujishima, J. Phys. Chem. 100, 48, 1996, 18603.

11. I. Samec. W. Johnson, M. Cappadonia, M.Jauch, K. Doblhofer, Sensors and Actuators, B, 13-14 (1993) 741.

12. S. Lundgren, B. Kasemo,Rev. Sci.Instrum. 66, 7, (1995) 3976.

13. C. S. Kumar, A. Subrahmanyam, J. Majhi, Rev. Sci. Instrum. 67 (3) (1996), 805.

14. H. A. Engelhardt, P. Feulner, H. Pfnür, D. Menzel, J. Phys E: Sci. Instr. 19, (1977), 1133.

15. I. D. Baikie, G. H. Bruggink, Mat. Res. Soc. Symp. Proc. 309, (1993), 35.

16. M. E. McGovern, M. Thompson, Can.J. Chem. 77 (1999), 1678.

17. L. M. Furtado, H.Su, M. Thompson, D. P. Mack, G. L. Hayword, Anal. Chem. 7, (1999), 1167.

18. M. E. McGovern, M. Thompson, Anal. Chem. Submitted.

19. 20. M. Nonnenmacher, M. P. O'Boyle and H. K. Wickramasinghe, Appl. Phys, Lett., 1991, 58, 25, 2921.

20. M. Nonnenmacher, M. P. O'Boyle and H. K. Wickramasinghe, Ultramicroscopy, 1992,42-44, 268.

21. M.Yasutake, J. Appl. Phys., 1995, 34, 3403.

22. M.Yasutake, A.Daisuke and M. Fujihira, Thin Solid Films, 1996, 723, 279.

The above-described embodiments of the present invention are intended to be examples only. Alterations, modifications and variations may be effected to the particular embodiments by those of skill in the art without departing from the scope of the invention, which is defined solely by the claims appended hereto.

What is claimed is:

1. A scanning Kelvin microprobe system for analyzing a biomolecular interaction on a surface of a substrate, said surface being capable of interacting with a biomolecule, the system comprising:
a tip with a predetermined work function for exploring the surface, and for extracting Kelvin current from the local capacitor formed between the tip and the substrate;
a scan table for placing the substrate thereon;
a micropositioner for moving the scan table in x and y directions;
a piezoelectric translation stage attached to the scan table for moving the substrate in the z direction for maintaining a constant substrate-tip distance;
a charge amplifier for converting the Kelvin current extracted by the tip into a voltage;
a first lock-in amplifier tuned at a first frequency for measuring the voltage and generating a contact potential difference image signal;
a second lock-in amplifier tuned at a second frequency for monitoring substrate-tip distance and for generating a topographic image signal, the second frequency being above the first frequency; and
a controller for controlling the micropositioner.

2. The scanning Kelvin microprobe system according to claim 1, additionally comprising a data acquisition system for acquiring said contact potential difference image signal and said topographical image signal.

3. The scanning Kelvin microprobe system according to claim 1, wherein said biomolecular interaction on a surface of a substrate comprises binding, hybridization, absorption, or adsorption.

4. The scanning Kelvin microprobe system according to claim 1, wherein the tip is a microelectrode having the apex radius of curvature less than about 100 nm.

5. The scanning Kelvin microprobe system according to claim 4, wherein said radius of curvature is about 50 nm.

6. The scanning Kelvin microprobe system according to claim 1, wherein the first frequency is from about 1 to about 20 kHz.

7. The scanning Kelvin microprobe system according to claim 1, wherein the second frequency is from about 100 to about 500 kHz.

8. The scanning Kelvin microprobe system according to claim 1, additionally comprising a substrate having a surface on which a probe is attached

9. A process for analyzing a biomolecular interaction on a surface of a substrate using a scanning Kelvin microprobe system, said surface being capable of interacting with a biomolecule, the process comprising the steps of:
placing a substrate on a scan table;
exploring a surface of the substrate with a tip having a predetermined work function;
extracting Kelvin current from a local capacitor formed between the tip and the substrate;
amplifying the Kelvin current extracted by the tip;
measuring the Kelvin current and generating a contact potential difference signal using a first lock-in amplifier tuned at a first frequency; and
monitoring distance between the substrate and the tip and generating a topographic image signal using a second lock-in amplifier tuned at a second frequency, the second frequency being above the first frequency.

10. The process according to claim 9, wherein the steps are controlled using a software program.

11. The process according to claim 9, wherein said biomolecular interaction on a surface of a substrate comprises binding, hybridization, absorption, or adsorption.

12. The process according to claim 9, wherein the first frequency is from about 1 to about 20 kHz.

13. The process according to claim 9, wherein the second frequency is from about 100 to about 500 kHz.

14. Use of a scanning Kelvin microprobe system according to any one of claims 1 to 8, for assessing biomolecular interaction on a substrate.

15. A process for analyzing an interaction between a probe and a target using a scanning Kelvin microprobe system according to any one of claims 1 to 8, comprising the steps of:
immobilizing a probe on the surface of a substrate;
subjecting the probe to a first scanning Kelvin microprobe analysis;
exposing the probe to a composition suspected of containing the target;
subjecting the substrate to a second scanning Kelvin microprobe analysis; and
comparing the results of the first and second scanning Kelvin microprobe analyses to determine interaction between the probe and the target.

16. The process according to claim 15, wherein at least one of the probe and the target is a nucleic acid, a polypeptide, or a small molecule.

17. The process according to claim 16, wherein at least one of the probe and the target is a small molecule.

18. The process according to claim 15, wherein at least one of the probe and the target is an antibody.

19. The process of claim 18, wherein one of the probe and the target is an antigen.
